(19) United States
(12) Patent Application Publication
(10) Pub. No.: US 2017/0258307 A1
(43) Pub. Date: Sep. 14, 2017

(54) ENDOSCOPE APPARATUS

(71) Applicant: OLYMPUS CORPORATION, Tokyo (JP)

(72) Inventors: Bakusui DAIDOJI, Hachioji-shi (JP); Eiji YAMAMOTO, Musashimurayama-shi (JP)

(73) Assignee: OLYMPUS CORPORATION, Tokyo (JP)

(21) Appl. No.: 15/604,987

(22) Filed: May 25, 2017

Related U.S. Application Data
(63) Continuation of application No. PCT/JP2014/081416, filed on Nov. 27, 2014.

(51) Int. Cl. A61B 1/06; A61B 1/04; A61B 1/00

(52) U.S. Cl. CPC A61B 1/063; A61B 1/00006; A61B 1/04; A61B 1/0638

(57) ABSTRACT

An endoscope apparatus includes a laser diode, an illumination section, an image sensor and a light source control section. The light source control section supplies the laser diode with different drive currents in sequence within exposure time of the image sensor to emit a plurality of laser lights in sequence from the laser diode and which controls the drive currents in such a manner that a combined wavelength spectrum width of combined laser light obtained by combining the emitted laser lights with the exposure time is made greater than a wavelength spectrum width of each of the laser lights.

### CROSS-REFERENCE TO RELATED APPLICATIONS

This application is a Continuation Application of PCT Application No. PCT/JP2014/081416, filed Nov. 27, 2014, the entire contents of all of which are incorporated herein by references.

### BACKGROUND OF THE INVENTION

1. Field of the Invention

The present invention relates to an endoscope apparatus that irradiates an observation target with light emitted from a laser diode as illumination light.

2. Description of the Related Art

In recent years, illumination apparatuses using a semiconductor laser have been actively developed. The illumination apparatuses using a semiconductor laser have the advantages of small size, high brightness and low power consumption, whereas they cause speckles due to high coherence of laser light.

The speckles are interference pattern caused by irradiating an object with light having high coherence such as laser light to reflect the light on the surface of the object and overlap the phases of scattered light, and the interference pattern reflect a state of the vicinity of the surface of the object. Since the speckles cause deterioration of the image quality, technology for reducing the speckles is under development.

The speckle reducing technology is disclosed in, for example, Jpn. Pat. Appln. KOKAI Publication No. 2008-253736. Jpn. Pat. Appln. KOKAI Publication No. 2008-253736 discloses a light-emitting device comprising a light-emitting unit including an excitation light source formed of a laser diode and a wavelength conversion member, and image sensor, in which the light-emitting unit is supplied with pulse current in a period not longer than the exposure time of the image sensor to vary a value of the current inputted to the light-emitting unit within the exposure time.

### BRIEF SUMMARY OF THE INVENTION

An endoscope apparatus according to the present invention comprises a laser diode, an illumination section which irradiates an observation target with laser light emitted from the laser diode as illumination light, an image sensor which images the observation target irradiated with the illumination light by the illumination section, and a light source controller which supplies the laser diode with different drive currents in sequence within exposure time of the image sensor to emit a plurality of laser lights in sequence from the laser diode.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute apart of the specification, illustrate embodiments of the invention, and together with the general description given above and the detailed description of the embodiments given below, serve to explain the principles of the invention.

FIG. 1 is a schematic configuration diagram showing an endoscope system to which an endoscope apparatus of the present invention is applied.

FIG. 2 is a block diagram showing one embodiment of the endoscope apparatus in the endoscope system.

FIG. 3 is a configuration diagram showing an optical diffusion unit.

FIG. 4 is a schematic graph showing the relationship between the pulse drive current of the first LD and the intensity of laser light, the wavelength spectrum width, and three pulse drive currents to be specified.

FIG. 5 is a graph showing a combined wavelength spectrum obtained by combining three pulsed lights emitted from the first LD within exposure time.

FIG. 6 is a schematic graph showing a mode hopping current.

FIG. 7 is a chart showing a specifying method according to two peak current values.

FIG. 8 is a chart showing a specifying method according to four peak current values.

FIG. 9 is a schematic graph showing a first light control method executed by pulse width control to control the light-emission time of an LD.

FIG. 10 is a schematic graph showing a second light control method to make pulse width control periods variable.

FIG. 11 is a schematic graph showing a third light control method executed by pulse width control when a duty ratio is decreased.

FIG. 12 is a schematic graph showing the third light control method executed by pulse width control when the duty ratio is increased.

FIG. 13 is a schematic graph showing a fourth light control method executed by pulse number control to control the number of pulses during a pulse number control period.

FIG. 14 is a schematic graph showing a fifth light control method executed by pulse number control to make a pulse number control period variable.

FIG. 15 is a schematic graph showing a sixth light control method executed by pulse number control when a duty ratio is decreased.

FIG. 16 is a schematic graph showing the sixth light control method executed by pulse number control when the duty ratio is increased.

FIG. 17 is a block diagram showing an endoscope apparatus according to a second variant.

FIG. 18A is a diagram showing a triangular waveform pulse drive current according to a third variant.

FIG. 18B is a diagram showing a saw-toothed waveform pulse drive current according to the third variant.

FIG. 18C is a diagram showing a curved waveform pulse drive current according to the third variant.

FIG. 19A is a graph showing a sine wave pulse drive current according to a fourth variant.

FIG. 19B is a graph showing a rectangular wave pulse drive current according to the fourth variant.

### DETAILED DESCRIPTION OF THE INVENTION

### One Embodiment

An endoscope apparatus according to one embodiment will be described below with reference to the drawings.

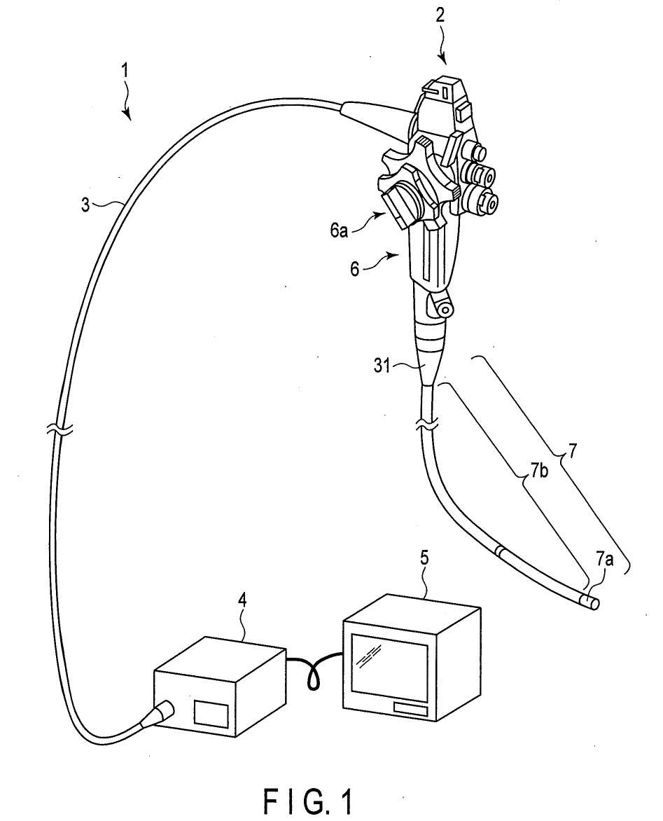

FIG. 1 is a schematic configuration diagram of an endoscope system 1 to which an endoscope apparatus is applied. The endoscope system 1 includes a scope unit 2, a cable 3, an endoscope main body 4 connected to the scope unit 2 through the cable 3, and an image display 5 connected to the endoscope main body 4.

The scope unit 2 includes the cable 3, an operation section 6 and an insertion section 7 coupled to the operation section 6. The operation section 6 includes an operation handle 6a. The operation handle 6a is intended to bend the insertion section 7 in up-and-down directions or right-and-left directions in response to an operator's operation.

The insertion section 7 is intended to observe an observation target in an object to be observed by inserting it into, for example, a lumen of the object to be observed. The insertion section 7 includes an insertion distal-end portion 7a that is formed rigidly and the other portion (referred to as an insertion bending portion hereinafter) 7b that is formed flexibly. Thus, the insertion bending portion 7b is passively bendable and if it is inserted into, for example, a lumen of an object to be observed, it is bent along the shape of the lumen. The insertion section 7 is also bent in the vertical or horizontal direction by the operation of the operation section 6. In other words, the insertion section 7 can be bent actively.

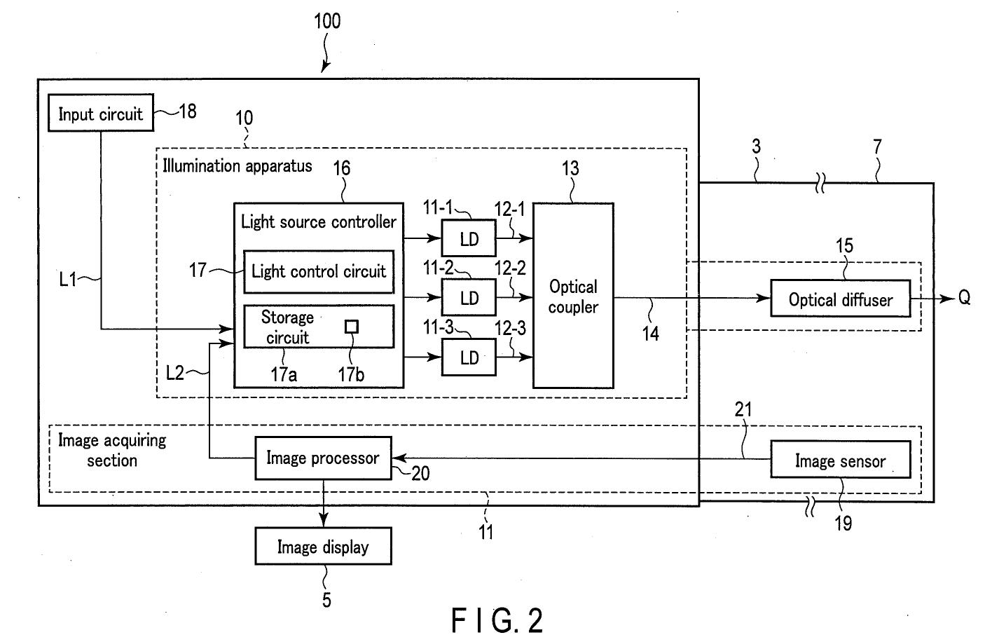

FIG. 2 is a block diagram of an endoscope apparatus 100 in the endoscope system 1. The endoscope main body 4 includes an illumination apparatus 10 that irradiates an observation target with illumination light and an image acquiring section 11 that acquires an image of the observation target. The image display 5 that displays an image of the observation target is connected to the image acquiring section 11.

The illumination apparatus 10 includes a plurality of laser diodes (referred to as LD hereinafter), e.g. three first to third LDs 11-1 to 11-3, first to third optical fibers 12-1 to 12-3, an optical coupler (referred to as optical fiber combiner hereinafter) 13, a fourth optical fiber 14, an optical diffuser 15 and a light source controller 16.

The first to third LDs 11-1 to 11-3 oscillate at different oscillation wavelengths and emit laser light. For example, the first LD 11-1 emits blue laser light whose central wavelength is 445 nm, the second LD 11-2 emits green laser light whose central wavelength is 532 nm, and the third LD 11-3 emits red laser light whose central wavelength is 635 nm.

The first optical fiber 12-1 optically connects the first LD 11-1 and the optical fiber combiner 13 and guides blue laser light emitted from the first LD 11-1 to the optical fiber combiner 13.

The second optical fiber 12-2 optically connects the second LD 11-2 and the optical fiber combiner 13 and guides green laser light emitted from the second LD 11-2 to the optical fiber combiner 13.

The third optical fiber 12-3 optically connects the first LD 11-3 and the optical fiber combiner 13 and guides red laser light emitted from the third LD 11-3 to the optical fiber combiner 13.

The optical fiber combiner 13 combines the blue laser light, green laser light and red laser light guided by the first optical fiber 12-1, second optical fiber 12-2 and third optical fiber 12-3, respectively into white laser light.

The fourth optical fiber 14 guides the white laser light combined by the optical fiber combiner 13 to the optical diffuser 15.

The first to third optical fibers 12-1 to 12-3 and the fourth optical fiber 14 are each a single fiber whose core diameter is, for example, several tens of μm to several hundreds of μm.

Coupling lenses (not shown) are provided between the first to third optical fibers 12-1 to 12-3 and the fourth optical fiber 12-4 in the optical fiber combiner 13. The coupling lenses cause blue laser light, green laser light and red laser light emitted from the first to third optical fibers 12-1 to 12-3 to converge, and couple them to the fourth optical fiber 12-4.

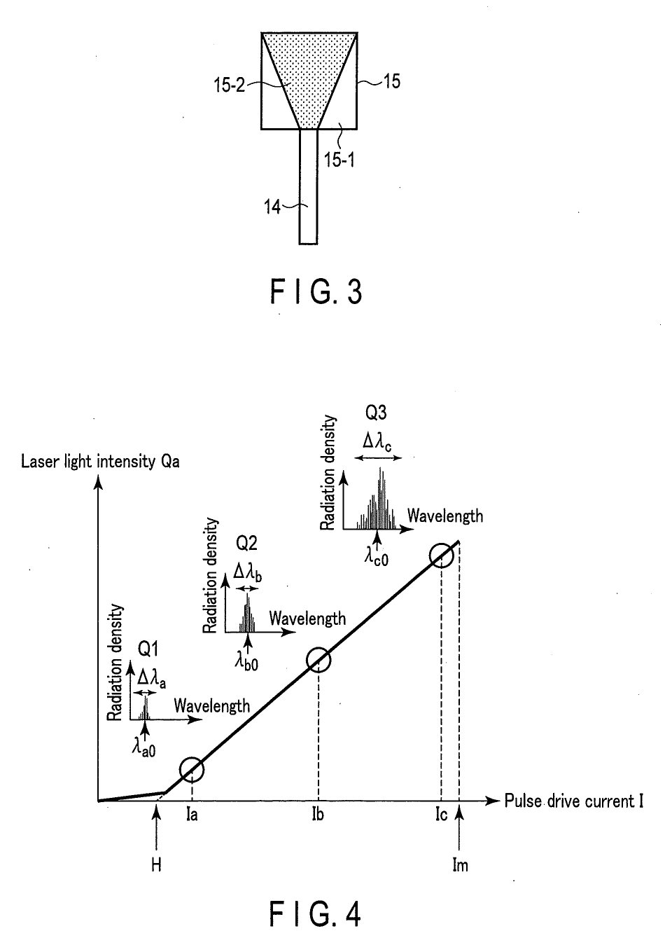

FIG. 3 is a configuration diagram of the optical diffuser 15. The optical diffuser 15 diffuses the white laser light guided by the fourth optical fiber 14. The white laser light diffused by the optical diffuser 15 is emitted as illumination light Q. The optical diffuser 15 includes a holder 15-1 and a diffusion member 15-2 such as an alumina particle, which is contained in the holder 15-1. The optical diffusion of the optical diffuser 15 brings about the advantage of widening the distribution of the white laser light guided by the fourth optical fiber 14 and disturbing the phase of the white laser beam to reduce the coherence and reduce the speckles.

The optical fiber 14 and optical diffuser 15 can be replaced with a bundle fiber including a plurality of optical fibers, e.g. several hundreds to several thousands of optical fibers and an illumination optical system (lens). The bundle fiber has an advantage of disturbing the phase of laser light emitted from the LDs to reduce speckles.

Pulse driving of a single one of the first to third LDs 11-1 to 11-3, e.g. the first LD 11-1, will be described below. The same pulse driving is true of the other LDs 11-2 and 11-3.

The light source controller 16 includes a light control circuit 17 to control the light of the first LD 11-1. The light control circuit 17 turns on and off the first LD 11-1 and controls the light intensity of the first LD 11-1.

The light source controller 16 supplies the first LD 11-1 with three pulse drive currents I whose peak currents are different from one another within exposure time to acquire a one-frame image by imaging of an image sensor 19 included in the image acquiring section 11. Since the image sensor 19 performs imaging periodically for each frame, the light source controller 16 supplies the first LD 11-1 with three pulse drive currents I periodically within the exposure time of the image sensor 19 to cause the first LD 11-1 to emit laser light.

FIG. 4 is a schematic graph showing the relationship between pulse drive current I of the first LD 11-1 and the intensity Qa of laser light. FIG. 4 also shows wavelength spectrum widths Δλa, Δλb and Δλc corresponding to three peak current values Ia, Ib and Ic of pulse drive currents I. The relationship in amount among the peak current values Ia, Ib and Ic is Ia<Ib<Ic. The central wavelengths of the wavelength spectrum widths Δλa, Δλb and Δλc are λa0, λb0 and λc0, respectively.

In the first LD 11-1, not only the laser light intensity Qa increases as the pulse drive current I increases, but also the wavelength spectrum widths Δλa, Δλb and Δλc widen as the oscillation mode increases. The central wavelengths λa0, λb0 and λc0 of the wavelength spectrum widths Δλa, Δλb and Δλc has a property to shift toward the long wavelength side. Accordingly, the relationship among the wavelength spectrum widths Δλa, Δλb and Δλc is given by the following:

Δλa<Δλb<Δλc

The relationship in size among the central wavelengths λa0, λb0 and λc0 of the wavelength spectrum widths Δλa, Δλb and Δλc is given by the following:

λa0≦λb0≦λc0

Since three pulse drive currents I, or pulse drive current I having three peak current values Ia, Ib and Ic is supplied to the first LD 11-1 within the exposure time of the image sensor 19, the wavelength spectra of three pulsed lights Q1, Q2 and Q3 emitted from the first LD 11-1 are combined within the exposure time. The width Δλabc (=Δλa+Δλb+Δλc) of the combined wavelength spectra is greater than each of the wavelength spectrum widths Δλa, Δλb and Δλc of three pulsed lights Q1, Q2 and Q3.

Therefore, the light source controller 16 supplies the first LD 11-1 with pulse drive current I having three peak current values Ia, Ib and Ic within the exposure time of the image sensor 19 to cause the first LD 11-1 to emit three pulsed lights Q1, Q2 and Q3.

The light source controller 16 controls the pulse drive current I in such a manner that the width Δλabc (=Δλa+Δλb+Δλc) of the combined wavelength spectrum of combined laser light Q obtained by combining the three pulsed lights Q1, Q2 and Q3 becomes greater than each of the wavelength spectrum widths Δλa, Δλb and Δλc of the three pulsed lights Q1, Q2 and Q3.

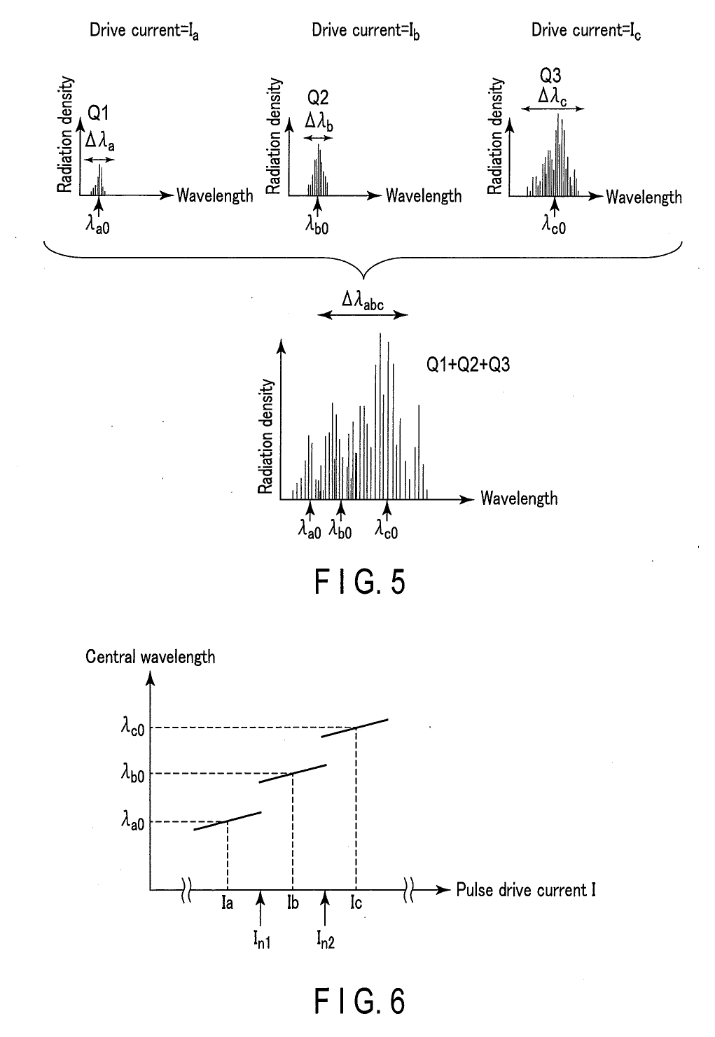

FIG. 5 shows a combined wavelength spectrum obtained by combining three pulsed lights Q1, Q2 and Q3 emitted from the first LD 11-1 within exposure time Tp. As shown in FIG. 5, as the pulse drive current I increases, the oscillation mode increases, the wavelength spectrum widths Δλa, Δλb and Δλc widen, and the central wavelengths λa0, λb0 and λc0 of the wavelength spectrum widths Δλa, Δλb and Δλc shift toward the long wavelength side.

Next, a method for specifying the three peak current values Ia, Ib and Ic of pulse drive current I will be described below.

The light source controller 16 includes a storage circuit 17a. The storage circuit 17a stores the three peak current values Ia, Ib and Ic of pulse drive currents I. There are first to fourth methods for specifying the three peak current values Ia, Ib and Ic.

(a) First Specifying Method

As shown in FIG. 4, the peak current value Ia is specified so that it is close to a lasing threshold current value H of the first LD 11-1 at the pulse drive current I and which is equal to or larger than the lasing threshold current value H (the specified peak current value is referred to as an lasing threshold neighboring current value). The lasing threshold neighboring current value is not larger 20% or more with reference to the lasing threshold current value H of the first LD 11-1. Further, even though the lasing threshold current value varies with a change in temperature of the first LD 11-1 and the like, the lasing threshold neighboring current value does not fall below the lasing threshold current value H but is specified as a current value that allows laser to be emitted stably.

The peak current value Ic is specified so that as it is close to the maximum rated current value Im of the first LD 11-1 and which is equal to or smaller than the maximum rated current value Im (the specified peak current value is referred to as a maximum rated neighboring current value). The maximum rated current value Im is the maximum value of a current value capable of being inputted to the first LD 11-1 safely. The maximum rated neighboring current value Ia is not less than 80% of the maximum rated current value Im and is specified as a current value having a given safety margin in consideration of variations due to a change in temperature of the first LD 11-1 and the like.

The peak current value Ib is specified as a value close to the average value between the lasing threshold neighboring current value and the maximum rated neighboring current value. Though it is better that the peak current value Ib is the intermediate current value between the lasing threshold neighboring current value and the maximum rated neighboring current value, it is desirable to specify the peak current value Ib at substantially an equal interval from the lasing threshold neighboring current value and the maximum rated neighboring current value in the current range. The intermediate current value represents a current value in the current range of not less than 20% with reference to the lasing threshold current value H of the first LD 11-1 and not more than 80% with reference to the maximum rated current value Im.

The light source controller 16 may specify one or both of the lasing threshold neighboring current value which is close to the lasing threshold current value H of the first LD 11-1 and which is equal to or larger than the lasing threshold current value H and the maximum rated neighboring current value which is close to the maximum rated current value Im of the first LD 11-1 and which is equal to or smaller than the maximum rated current value Im, as the peak current values Ia and Ic of pulse drive current I.

The light source controller 16 may specify the lasing threshold neighboring current value, the rated current neighboring current value, and the intermediate current value between the lasing threshold neighboring current value and the rated current neighboring current value, as the peak current values Ia, Ib and Ic of pulse drive current I.

The light source controller 16 may specify the peak current values Ia, Ib and Ic of pulse drive current I at an equal interval from the lasing threshold neighboring current value and the rated current neighboring current value in the current range. The central wavelength λ0 of the wavelength spectrum of pulsed lights Q1, Q2 and Q3 emitted from the first LD 11-1 shifts toward the long wavelength side as the pulse drive current I becomes larger. It is thus desirable to specify the peak current values Ia, Ib and Ic of pulse drive current I at substantially an equal interval in the wide current range as described above.

The three peak current values Ia, Ib and Ic are each specified over the mode hopping current values of the first LD 11-1. The mode hopping current values are pulse drive current values Ih1 and Ih2 obtained when the oscillation mode varies discontinuously when the pulse drive current I of the first LD 11-1 varies continuously as shown in, e.g. FIG. 6. For example, if the pulse drive current values Ih1 and Ih2 are obtained when the pulse drive current I increases continuously, the wavelength λ of each pulsed light emitted from the first LD 11-1 jumps to a short wavelength value. On the other hand, if the pulse drive current values Ih1 and Ih2 are obtained when the pulse drive current I decreases continuously, the wavelength A of each pulsed light emitted from the first LD 11-1 jumps to a long wavelength vale. In other words, the mode hopping means that when the peak current values are specified over the mode hopping currents Ih1 and Ih2, the central wavelengths λa0, λb0 and λc0 of the wavelength spectrum widths Δλa, Δλb and Δλc jump discontinuously. The mode hopping occurs due to a change in gain peak, a change in lateral mode or the like when the refractive index varies as the internal temperature of the first LD 11-1 varies.

If the three peak current values Ia, Ib and Ic of pulse drive currents I are specified over the mode hopping currents Ih1 and Ih2 as described above, the wavelength region of the wavelength spectrum of one (Q1) of any two (e.g. Q1 and Q2) of three pulsed lights Q1, Q2 and Q3 is not included in the wavelength region of the wavelength spectrum of the other pulsed light Q2.

Therefore, the light source controller 16 controls the three peak current values Ia, Ib and Ic of pulse drive currents I in such a manner that the central wavelengths of two of a plurality of pulsed lights, e.g. three pulsed lights Q1, Q2 and Q3, which are adjacent with respect to the wavelength axis, e.g. the central wavelengths λa0 and λb0 of the pulsed lights Q1 and Q2 have a wavelength difference that is equal to or greater than a given wavelength difference based on the wavelength spectrum widths Δλa and Δλb of the two pulsed lights.

The light source controller 16 may control the three peak current values Ia, Ib and Ic of pulse drive currents I in such a manner that the wavelength region of the wavelength spectrum of one of a plurality of pulsed lights, e.g. three pulsed lights Q1, Q2 and Q3 is not included in the wavelength region of the wavelength spectrum of the other pulsed lights.

Furthermore, the light source controller 16 may control the three peak current values in such a manner that the central wavelengths of adjacent two of the three pulsed lights Q1, Q2 and Q3, e.g. the central wavelengths of the pulsed lights Q1 and Q2 have a wavelength difference that is equal to or greater than the sum of halves of the wavelength spectrum widths Δλa and Δλb of the two pulsed lights Q1 and Q2.

Therefore, the light source controller 16 controls the three peak current values Ia, Ib and Ic of pulse drive currents I to have a wavelength difference that is equal to or greater than the sum of halves of the wavelength spectrum widths Δλa and Δλb of two pulsed lights adjacent with respect to the wavelength axis, e.g. pulsed lights Q1 and Q2 of a plurality of pulsed lights, e.g. three pulsed lights Q1, Q2 and Q3.

As a result, the combined wavelength spectrum width Δλabc (=Δλa+Δλb+Δλc) obtained within exposure time Tp of the image sensor 19 can effectively be made greater than each of the wavelength spectrum widths Δλa, Δλb and Δλc. Thus, coherence is lowered and speckles can effectively be reduced.

(b) Second Specifying Method

The lasing threshold neighboring current value and the maximum rated neighboring current value are specified as in the foregoing first specifying method. These current values are defined as peak current values Ia and Ic, respectively.

A peak current value Ibis specified in such a manner that the central wavelength λb0 of pulsed light Q2 exists near the average value between the central wavelength λa0 of pulsed light Q1 whose lasing threshold neighboring current value is peak current Ia and the central wavelength λc0 of pulsed light Q3 whose maximum rated neighboring current value is peak current Ic.

Thus, the light source controller 16 specifies an intermediate current value in such a manner that the central wavelength λc0 of pulsed light Q2 exists at an equal interval from the central wavelength λa0 of pulsed light Q1 and the central wavelength λc0 of pulsed light Q3 in the wavelength range between the central wavelength λa0 of pulsed light Q1 whose lasing threshold neighboring current value is a peak current and the central wavelength λc0 of pulsed light Q3 whose maximum rated neighboring current value is peak current Ic.

In this case, the peak current Ib is specified after the peak current dependency of pulse on the central wavelength is measured, like the central wavelength λa0 of pulsed light Q1 whose lasing threshold neighboring current value is a peak current and the central wavelength λb0 of pulsed light Q2 whose rated neighboring current value is peak current Ib.

(c) Third Specifying Method

In the first and second specifying methods, the pulse drive current I has, for example, three peak current values Ia, Ib and Ic; however, it may have two peak current values or four or more peak current values.

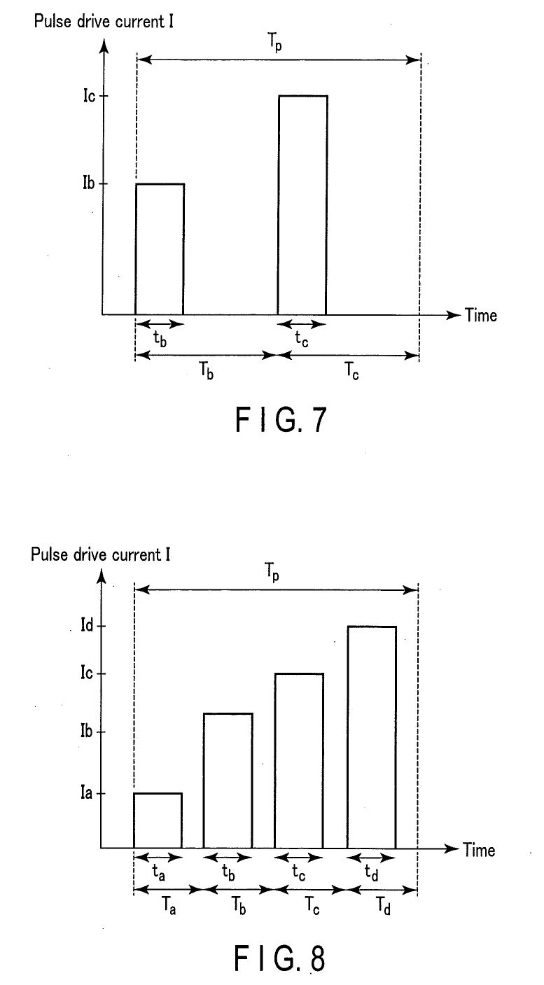

FIG. 7 shows a specifying method according to two peak current values. Two peak current values Ib and Ic are generated within exposure time Tp to acquire a one-frame image by imaging of the image sensor 19. The peak current values Ib and Ic have pulse widths tb and tc, respectively. The peak current values Ib and Ic are repeatedly generated at intervals of Tb and Tc (=Tb), respectively.

FIG. 8 shows a specifying method according to four peak current values. Four peak current values Ia to Id are generated within exposure time Tp to acquire a one-frame image by imaging of the image sensor 19. The peak current values Ia to Id have pulse widths to to td, respectively. The peak current values Ia to Id are repeatedly generated at intervals of Ta to Td (Ta=Tb=Tc=Td), respectively.

In a specifying method of according to four or more peak current values, the peak current values of pulse drive currents I are specified at substantially an equal interval from the current value close to lasing threshold current value and the current value close to maximum rated current value in the current range. The four peak current values Ia to Id shown in FIG. 8 are specified at equal intervals of Ta to Td (Ta=Tb=Tc=Td).

In the specifying method according to four or more peak current values, the central wavelengths of two pulsed lights adjacent with respect to the wavelength axis have a wavelength difference that is equal to or greater than half the wavelength spectrum width of one of the two pulsed lights, the wavelength spectrum width of which is smaller.

Therefore, the light source controller 16 controls the peak current values Ia to Id of a plurality of pulse drive currents I in such a manner that the central wavelengths of two of a plurality of pulsed lights, e.g. four or more peak current values, or the central wavelengths of, e.g. pulsed lights Q1 and Q2 adjacent with respect to the wavelength axis have a wavelength difference that is equal to or greater than half the wavelength spectrum width of one of the two pulsed lights Q1 and Q2, the wavelength spectrum width of which is smaller.

In the specifying method according to four or more peak current values, in either case, at least one of the lasing threshold neighboring current value and the maximum rated neighboring current value is specified.

In the specifying method according to four or more peak current values, the peak current values are specified over the mode hopping currents.

(d) Fourth Specifying Method

In the fourth specifying method, the central wavelengths λa0, λb0 and λc0 of pulsed lights Q1, Q2 and Q3 are measured in advance for three peak current values Ia, Ib and Ic of pulse drive currents I.

In the specifying method, the three peak current values Ia, Ib and Ic are specified in such a manner that the wavelength region of the wavelength spectrum of one of any two of the three pulsed lights Q1, Q2 and Q3 is not included in the wavelength region of the wavelength spectrum of the other pulsed light.

The central wavelengths of two of the three pulsed lights Q1, Q2 and Q3, which are adjacent to each other with respect to the wavelength axis, e.g. the central wavelengths λa0 and λb0 of the pulsed lights Q1 and Q2 have a wavelength difference that is equal to or greater than half the wavelength spectrum width Δλa or Δλb of one of the two pulsed lights Q1 and Q2. Favorably, the three peak current values Ia, Ib and Ic are specified to have a wavelength difference that is equal to or greater than the sum of halves of the wavelength spectrum widths Δλa and Δλb of the two pulsed lights Q1 and Q2.

Next, a light control method for the first to third LDs 11-1 to 11-3 will be described below.

An intensity of light emitted from the first to third LDs 11-1 to 11-3 is obtained based upon illumination light intensity control information L1 inputted to an input circuit 18 by a user's operation or illumination light intensity control information L2 calculated by image processing of an image processor 20 included in the image acquiring section 11. The image processor 20 calculates the illumination light intensity control information L2 by processing the brightness information of an image of an observation target.

The light source controller 16 includes the storage circuit 17a. The storage circuit 17a stores light intensity ratio information LI indicating the ratio of intensities of light emitted from the first to third LDs 11-1 to 11-3 such that illumination light Q has a desired color. The desired color is, for example, white light with high color rendering properties, such as the color of an object to be observed which is reproduced when light is emitted from a xenon lamp or a halogen lamp.

The light source controller 16 calculates an intensity of laser light emitted from each of the first to third LDs 11-1 to 11-3 on the basis of the illumination light intensity control information L1 or L2 and the light intensity ratio information LI.

The light source controller 16 includes the light control circuit 17 to control the light of the first to third LDs 11-1 to 11-3. The light control circuit 17 makes light control based upon the intensity of laser light emitted from each of the first to third LDs 11-1 to 11-3 and calculated by the light source controller 16.

A light control method for a single one of the first to third LDs 11-1 to 11-3, e.g. the first LD 11-1 will be described below. The same light control method is true of the other LD 11-2 and 11-3.

The following are first to sixth light control methods for the first LD 11-1.

(a) First Light Control Method

The light control circuit 17 controls the light of the first LD 11-1 by performing pulse width control to control the light-emission time of the first LD 11-1 within exposure time Tp for the three peak current values Ia, Ib and Ic, or pulse width control which controls the pulse widths of the peak current values Ia, Ib and Ic of pulse drive currents I.

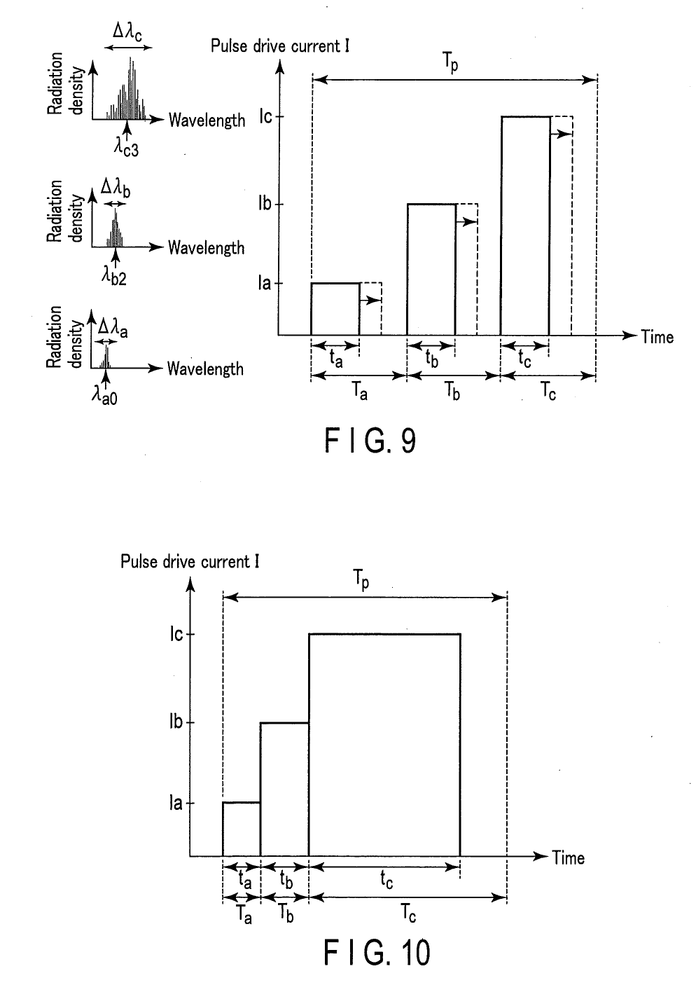

Specifically, the light control circuit 17 specifies pulse width control periods Ta, Tb and Tc for the three peak current values Ia, Ib and Ic of pulse drive currents I as shown in FIG. 9. The light control circuit 17 controls light-emission time ta of the first LD 11-1 within the pulse width control period Ta, controls light-emission time tb of the first LD 11-1 within the pulse width control period Tb, and controls light-emission time tc of the first LD 11-1 within the pulse width control period Tc. In other words, the ratio ta/Ta of light-emission time ta of the first LD 11-1 to the pulse width control period Ta, the ratio tb/Tb of light-emission time tb of the first LD 11-1 to the pulse width control period Tb, and the ratio tc/Tc of light-emission time tc of the first LD 11-1 to the pulse width control period Tc are duty ratios Da, Db and Dc, respectively. Here, the pulse width control periods Ta, Tb and Tc are fixed.

Thus, the light control circuit 17 controls the light of the first LD 11-1 by controlling each of the duty ratios Da, Db and Dc. FIG. 9 shows a state in which the light-emission times ta, tb and tc of the first LD 11-1 are lengthened to increase the duty ratios Da, Db and Dc.

The pulse width control periods Ta, Tb and Tc for the pulse drive currents I are fixed by time obtained by dividing the exposure time Tp by the number of peak current values Ia, Ib and Ic, or three here (Tp/3).

Specifically, the pulse width control of the light control circuit 17 is performed as follows. In the storage circuit 17a, a light control table 17b is stored. The light control table 17b has pulse width control information to control the light of the first LD 11-1, or pulse width control information to control the light-emission times ta, tb and tc of the first LD 11-1. This pulse width control information includes information indicating how the duty ratios Da, Db and Dc of the peak current values Ia, Ib and Ic are set for the intensity of laser light emitted from the first LD 11-1.

The light control circuit 17 controls the light intensity of the first LD 11-1 on the basis of information stored in the light control table 17b. In other words, to increase the intensity of laser light emitted from the first LD 11-1, the light control circuit 17 preferentially increases the duty ratio Da of pulse current I whose peak current values Ia, Ib and Ic are small.

To decrease the intensity of laser light emitted from the first LD 11-1, the light control circuit 17 preferentially decreases the duty ratio Dc of pulse drive current I whose peak current values Ic is large.

With the light control described above, the duty ratio Da of the pulse drive current I whose peak current Ia is small is secured as much as possible to prevent a contribution to the combined wavelength spectrum width Δλabc (=Δλa+Δλb+Δλc) from decreasing.

(b) Second Light Control Method

The light control circuit 17 makes the pulse width control periods variable with reference to the peak current values Ia, Ib and Ic in accordance with the intensity of laser light emitted from the first LD 11-1. FIG. 10 is a schematic graph showing a second light control method to make the pulse width control periods variable.

In the first light control method, if each of the duty ratios is maximized for exposure during the exposure time Tp, the light intensity cannot be increased further. If a further light intensity (large light intensity) is included, the second light control method is employed as a large light intensity mode.

When the intensity of laser light emitted from the first LD 11-1 is large, the light control circuit 17 makes the pulse width control periods Ta, Tb and Tc longer as the peak current of pulse current increases. In FIG. 10, the pulse width control period Tc of peak current Ic is controlled long. To reduce the light intensity from this state, the light control circuit 17 lengthens the pulse width control periods from the pulse current whose peak current is small.

If the pulse width control periods Ta, Tb and Tc are controlled to make alight control, the light control range can be broadened.

(c) Third Light Control Method

The light control circuit 17 fixes the ratio of pulse widths of three peak current values Ia, Ib and Ic, or the ratio of light-emission times of the first LD 11-1, and treats the three peak current values Ia, Ib and Ic as one pulse. In this state, the light control circuit 17 controls the duty ratio D within exposure time Tp to make a light control. The pulse width control periods Ta, Tb and Tc are not defined with reference to the peak current values Ia, Ib and Ic.

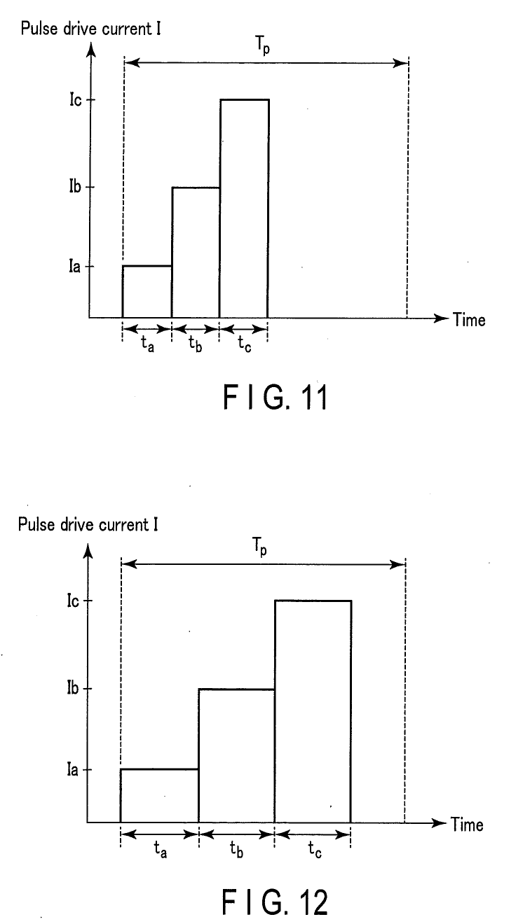

FIGS. 11 and 12 each show an example of making the light-emission times ta, tb and tc of the first LD 11-1 variable with the ratio of the light-emission times ta, tb and tc fixed. The light-emission times ta, tb and tc shown in FIG. 12 are controlled longer than the light-emission times ta, tb and tc shown in FIG. 11. Since the ratio of the light-emission times ta, tb and tc is fixed, the relationship among ta, tb and tc is maintained as ta=tb=tc.

(d) Fourth Light Control Method

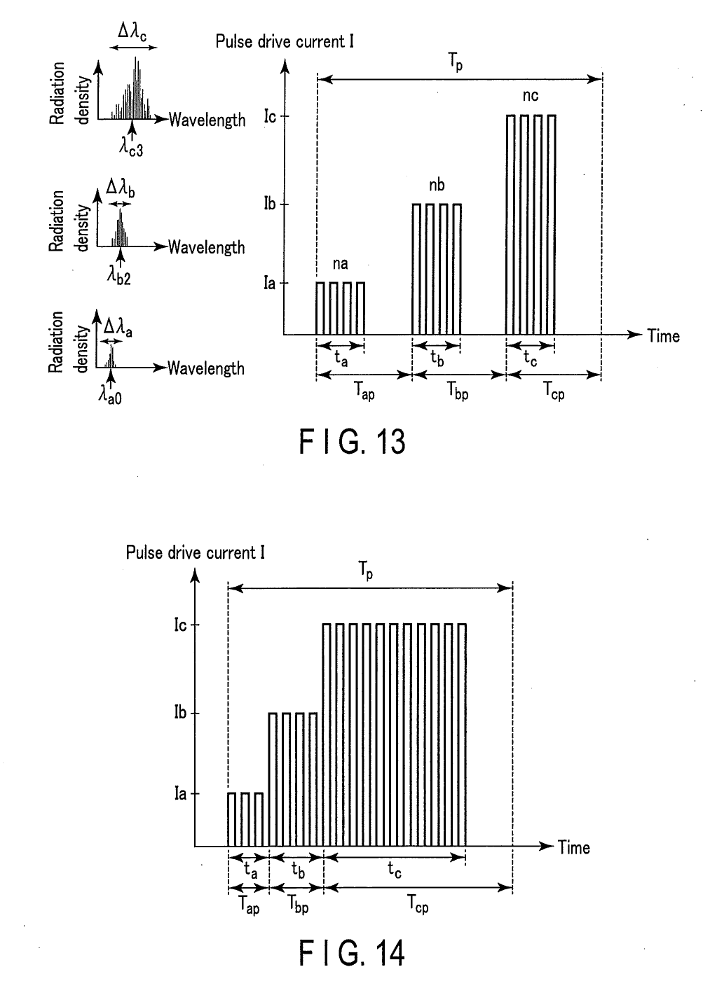

FIG. 13 shows a fourth light control method executed by pulse number control. As shown in FIG. 13, the light control circuit 17 sets predetermined periods, or pulse number control periods Tap, Tbp and Tcp within exposure time Tp. The light control circuit 17 makes a light control by generating a plurality of pulses of the same peak current value within each of the light-emission times ta, tb and tc of pulses of three peak current values Ia, Ib and Ic for each of the pulse number control periods Tap, Tbp and Tcp and controlling the numbers na, nb and nc of the pulses (pulse number control). The pulse number control periods Tap, Tbp and Tcp are fixed to time obtained by dividing the exposure time Tp by the number of peak current values Ia, Ib and Ic, or three here.

The light control circuit 17 controls the number of pulses on the basis of pulse number control information stored in advance in the light control table 17b of the storage circuit 17a. The light control table 17b stores, as the pulse number control information, for example, the pulse number control periods Tap, Tbp and Tcp corresponding to the three peak current values Ia, Ib and Ic, the periods of pulses generated during the pulse number control periods Tap, Tbp and Tcp, and the number of pulses generated within the light-emission times ta, tb and tc of the first LD 11-1 corresponding to the three peak current values Ia, Ib and Ic.

To increase the intensity of laser light emitted from the first LD 11-1, the light control circuit 17 preferentially increases the number of pulses within the light-emission time of a small peak current value, e.g. the pulse number na within the light-emission time ta of a small peak current value Ia.

To decrease the intensity of laser light emitted from the first LD 11-1, the light control circuit 17 preferentially decreases the number of pulses within the light-emission time of a large peak current value, e.g. the pulse number nc within the light-emission time tc of a large peak current value Ic.

With the light control described above, the pulse number (light intensity) na within the light-emission time ta of the first LD 11-1 by a small pulse drive current Ia whose peak current is small can be secured to prevent a contribution to the combined wavelength spectrum width Δλabc (=Δλa+Δλb+Δλc) from decreasing.

(e) Fifth Light Control Method

The light control circuit 17 makes a light control by making the pulse number control periods Tap, Tbp and Tcp variable in accordance with the intensity of laser light emitted from the first LD 11-1 to control the number of pulses and making the pulse width control periods Ta, Tb and Tc variable in proportion to the amount of the three peak current values Ia, Ib and Ic if the intensity of laser light emitted from the first LD 11-1 is larger than a predetermined light intensity.

In other words, the fifth light control method makes it possible to control the pulse number control periods Tap, Tbp and Tcp for the three peak current values Ia, Ib and Ic in accordance with the intensity of laser light emitted from the first LD 11-1.

If the intensity of laser light emitted from the first LD 11-1 is small as shown in FIG. 14, the pulse number control periods Tap, Tbp and Tcp are shortened as the pulse drive current I decreases in peak current.

If the intensity of laser light emitted from the first LD 11-1 is large, the pulse number control periods Tap, Tbp and Tcp are lengthened as the pulse drive current I increases in peak current. In other words, the pulse number control periods Tap, Tbp and Tcp are shortened as the pulse drive current I decreases in peak current.

The foregoing light control allows the light control range to be broadened.

(f) Sixth Light Control Method

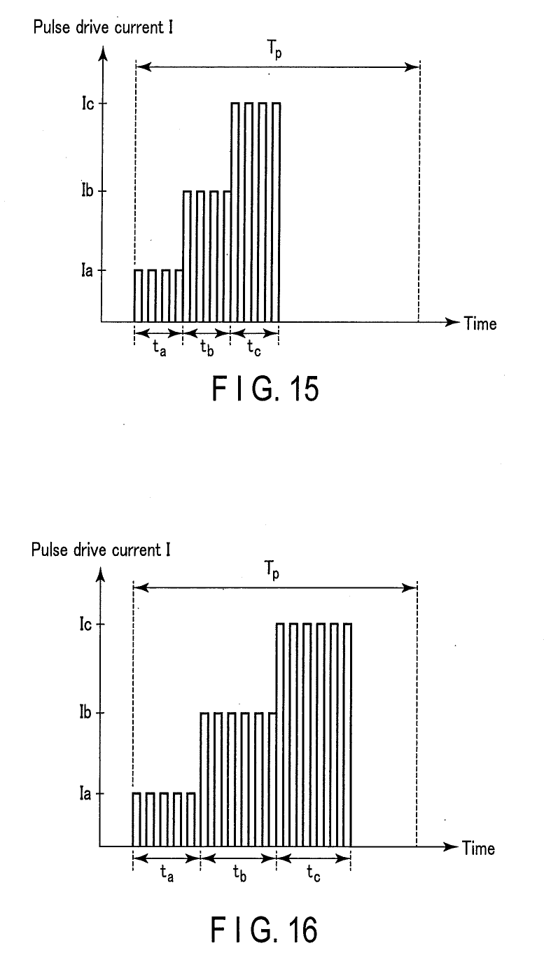

FIGS. 15 and 16 are schematic graphs showing a sixth light control method executed by pulse number control. In the sixth light control method, the ratio of pulse widths of three peak current values Ia, Ib and Ic is fixed to control the pulse number for each of the three peak current values Ia, Ib and Ic.

The light control circuit 17 fixes the ratio of pulse widths of three peak current values Ia, Ib and Ic and fixes the ratio of the number of pulses generated within light-emission times ta, tb and tc of the first LD 11-1. For example, the number of pulses generated within light-emission times ta, tb and tc of the first LD 11-1 shown in FIG. 15 and the number of pulses generated within light-emission times ta, tb and tc of the first LD 11-1 shown in FIG. 16 are proportional to lengths of the light-emission times ta, tb and tc.

The image acquiring section 11 includes the image sensor 19 and the image processor 20. The image sensor 19 and image processor 20 are connected through an imaging cable 21. The image sensor 19 receives a light image reflected from an observation target, images the observation target and outputs an imaging signal. Specifically, the image sensor 19 includes, e.g. a CCD imager, a CMOS imager. The frame rate of the image sensor 19 is, e.g. a frequency of 30 Hz (fps).

The image processor 20 receives an image signal from the image sensor 19 and processes the image signal to acquire an image of the observation target. The image processor 20 performs image processing on the basis of brightness information included in the image signal output from the image sensor 19 to calculate second light intensity control information L2. The second light intensity control information L2 is intended to cause the image of the observation target to have an appropriate brightness value and is sent to the light control circuit 17.

The image display 5 displays the image of the observation target which is acquired by the image processor 20. The image display 5 includes a monitor such as a liquid crystal display.

The input circuit 18 receives an operator's operation and outputs first light intensity control information L1 for illumination light Q. The first light intensity control information L1 is sent to the light control circuit 17 of the light source controller 16.

Next, an operation of the endoscopic illumination apparatus 10 configured as described above will be described below.

The input circuit 18 receives an operator's operation and outputs first light intensity control information L1 for illumination light Q.

The image processor 20 performs image processing on the basis of bright information included in the image signal output from the image sensor 19 to calculate second light intensity control information L2. The second light intensity control information L2 is intended to cause the image of an observation target to have an appropriate brightness value and is sent to the light source controller 16.

The light source controller 16 calculates an intensity of laser light emitted from the first to third LD 11-1 to 11-3 on the basis of the illumination light intensity control information L1 or L2 and the light intensity ratio information LI. The light control circuit 17 makes a light control on the basis of the intensity of laser light emitted from the first to third LD 11-1 to 11-3, which is calculated by the light source controller 16.

In this case, the light source controller 16 supplies the first LD 11-1 with the pulse drive currents I of three peak current values Ia, Ib and Ic within the exposure time of the image sensor 19 and causes the first LD 11-1 to emit three pulsed lights Q1, Q2 and Q3.

The light source controller 16 controls the pulse drive currents I in such a manner that the combined wavelength spectrum width Δλabc (=Δλa+Δλb+Δλc) of combined laser light Q into which the three pulsed lights Q1, Q2 and Q3 are combined, is made greater than each of the wavelength spectrum widths Δλa, Δλb and Δλc of the three pulsed lights Q1, Q2 and Q3.

The three peak current values Ia, Ib and Ic are specified by the foregoing first to fourth specifying methods.

In the first specifying method, as shown in FIG. 4, the peak current value Ia is specified as a value (the lasing threshold neighboring current value) which is close to the lasing threshold current value H of the first LD 11-1 at the pulse drive current I and which is equal to or larger than the lasing threshold current value H.

The peak current value Ic is specified as a value (the maximum rated neighboring current value) which is close to the maximum rated current value Im of the first LD 11-1 and which is equal to or smaller than the maximum rated current value Im.

The peak current value Ib is specified as a value close to the average value between the lasing threshold neighboring current value and the maximum rated neighboring current value.

In the second specifying method, the peak current values Ia and Ic are specified as in the first specifying method with respect to the lasing threshold neighboring current value and the maximum rated neighboring current value.

The peak current value Ib is specified in such a manner that the central wavelength λb0 of pulsed light Q2 exists near the average value between the central wavelength λa0 of pulsed light Q1 whose lasing threshold neighboring current value is peak current Ia and the central wavelength λc0 of pulsed light Q3 whose maximum rated neighboring current value is peak current Ic.

If the number of peak current values is not three, they are specified by the third specifying method.

In the fourth specifying method, the wavelength region of the wavelength spectrum of one of any two of three pulsed lights Q1, Q2 and Q3 is not included in the wavelength region of the wavelength spectrum of the other pulsed light. In the fourth specifying method, the three peak current values Ia, Ib and Ic are specified to have a wavelength difference that is equal to or greater than the sum of halves of the wavelength spectrum widths of the two pulsed lights.

The same is true of the other second LD 11-2 and third LD 11-3.

Furthermore, the light control circuit 17 of the light source controller 16 controls the light of the first to third LD 11-1 to 11-3 by one of the foregoing first to sixth light control methods.

In the first light control method, the light control circuit 17 performs pulse width control to control the light-emission time of the first LD 11-1 within exposure time Tp for three peak current values Ia, Ib and Ic, or pulse width control to control the pulse widths of the peak current values Ia, Ib and Ic of pulse drive currents I.

In the second light control method, the light control circuit 17 makes the pulse width control periods variable with reference to the peak current values Ia, Ib and Ic in accordance with the intensity of laser light emitted from the first LD 11-1.

In the third light control method, the light control circuit 17 fixes the ratio of pulse widths of three peak current values Ia, Ib and Ic, or the ratio of light-emission times ta, tb and tc of the first LD 11-1, and treats the three peak current values Ia, Ib and Ic as one pulse, thus controlling the duty ratio D within exposure time Tp.

In the fourth light control method, as shown in FIG. 13, the light control circuit 17 sets pulse number control periods Tap, Tbp and Tcp within exposure time Tp, generates a plurality of pulses of the same peak current value within each of the light-emission times ta, tb and tc of pulses of three peak current values Ia, Ib and Ic for each of the pulse number control periods Tap, Tbp and Tcp, and controls a plurality of pulse numbers na, nb and nc (pulse number control).

In the fifth light control method, the light control circuit 17 makes the pulse number control periods Tap, Tbp and Tcp variable in accordance with the intensity of laser light emitted from the first LD 11-1 and makes lengths of the pulse width control periods Ta, Tb and Tc variable in proportion to the amount of three peak current values Ia, Ib and Ic if the intensity of laser light emitted from the first LD 11-1 is larger than a predetermined light intensity.

In the sixth light control method, the light control circuit 17 fixes the ratio of pulse widths of three peak current values Ia, Ib and Ic to control the pulse number for each of the three peak current values Ia, Ib and Ic.

The same is true of the other second LD 11-2 and third LD 11-3.

The first to third LD 11-1 to 11-3 emit blue laser light, green laser light and red laser light whose light intensity are controlled. These blue, green and red laser lights are guided by their respective optical fibers 12-1, 12-2 and 12-3 and enter the optical fiber combiner 13. The optical fiber combiner 13 combines the blue, green and red laser lights and emits white laser light. The white laser light emitted from the optical fiber combiner 13 is guided by the optical fiber 14 and enters the optical diffuser 15.

The optical diffuser 15 diffuses the white laser light guided by the fourth optical fiber 14. The diffused white laser light is radiated to an observation target as illumination light Q.

The image sensor 19 receives light reflected from the observation target, images the observation target and then outputs an imaging signal.

The image processor 20 receives the image signal from the image sensor 19 and processes the image signal to acquire an image of the observation target. The image of the observation target is displayed on the image display 5.

The image processor 20 performs image processing on the basis of bright information included in the image signal output from the image sensor 19 to calculate second light intensity control information L2. The second light intensity control information L2 is sent to the light control circuit 17.

As described above, according to the foregoing one embodiment, the pulse drive currents I are so controlled that the combined wavelength spectrum width Δλabc (=Δλa+Δλb+Δλc) of combined laser light into which three pulsed lights Q1, Q2 and Q3 are combined, is made greater than each of the wavelength spectrum widths Δλa, Δλb and Δλc of the three pulsed lights Q1, Q2 and Q3.

Thus, the wavelength spectra of the three pulsed lights Q1, Q2 and Q3 emitted from the first LD 11-1 are combined within the exposure time. The combined wavelength spectrum width Δλabc (=Δλa+Δλb+Δλc) of the combined wavelength spectra is greater than each of the wavelength spectrum widths Δλa, Δλb and Δλc of the three pulsed lights Q1, Q2 and Q3.

The same is true of the other second LD 11-2 and third LD 11-3.

Therefore, the illumination light Q emitted from the optical diffuser 15 can be decreased in coherence. Speckles on an image acquired by imaging of the image sensor 19 can sufficiently be reduced. Accordingly, an image of an observation target in an object to be observed whose speckles are reduced can be observed.

[First Variant]

A first variant will be described below. In the foregoing one embodiment, an observation target is observed by emitting white illumination light Q from the three LDs 11-1 to 11-3. The number of LD is not limited to three, but four or more LDs can be used. The use of four or more LDs makes it possible to make an observation using white light with higher color rendering properties than the use of three LDs, an observation using two LD of a blue-violet LD and a green LD to emphatically display a blood vessel using light absorption characteristics of hemoglobin, and an observation using only one LD having a near-infrared wavelength.

[Second Variant]

A second variant will be described below. The same elements as those shown in FIG. 2 are denoted by the same reference numeral and their detailed descriptions will be omitted.

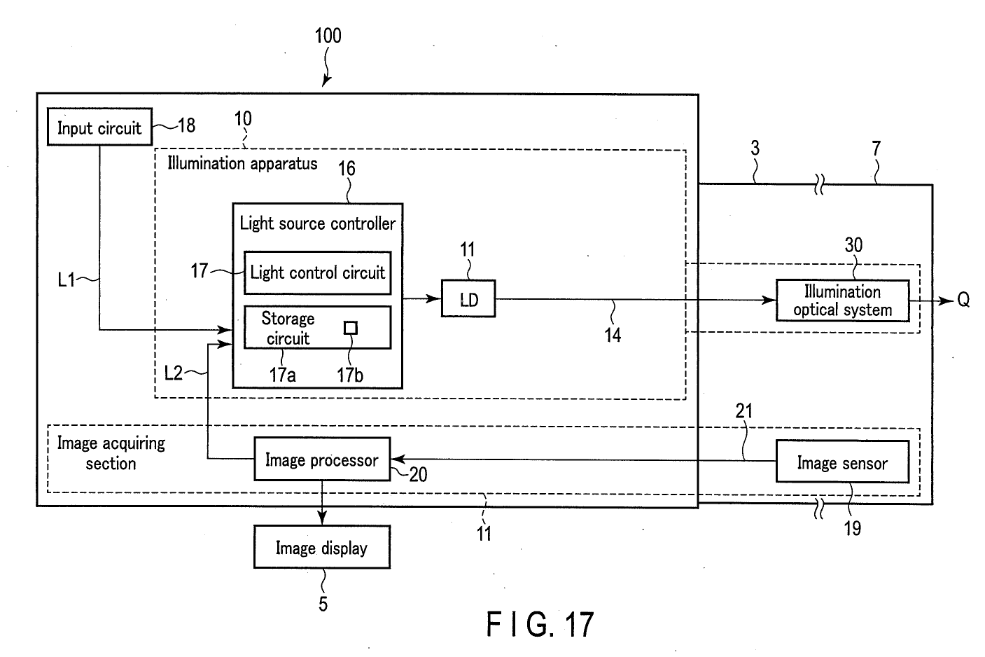

FIG. 17 is a block diagram showing an endoscopic illumination apparatus 10 according to the second variant.

The illumination apparatus 10 includes one LD 11. The LD 11 is, for example, one of first LD 11-1, second LD 11-2 and third LD 11-3, or another LD that emits laser light having a central wavelength.

The LD 11 is optically connected to the optical diffuser 15 through the optical fiber 14. Since one LD 11 is provided, the need for the optical fiber combiner 13 of the foregoing one embodiment is obviated.

The light source controller 16 calculates an intensity of laser light emitted from the LD 11 on the basis of the illumination light intensity control information L1 or L2 and the light intensity ratio information LI.

The light control circuit 17 makes a light control on the basis of the intensity of laser light emitted from the LD 11, which is calculated by the light source controller 16. The light control circuit 17 makes a light control for the LD 11 by one of the foregoing first to sixth light control methods.

An illumination optical system 30 is connected to the fourth optical fiber (simply referred to as an optical fiber). The illumination optical system 30 irradiates an observation target with laser light guided by the optical fiber 14 as illumination light Q.

The LD 11 emits, for example, blue, green or red laser light. This laser light is guided by the optical fiber 14 and enters the illumination optical system 30. The illumination optical system 30 irradiates an observation target with laser light guided by the optical fiber 14 as illumination light Q.

Needless to say, the second variant brings about the same advantages as those of the foregoing one embodiment.

[Third Variant]

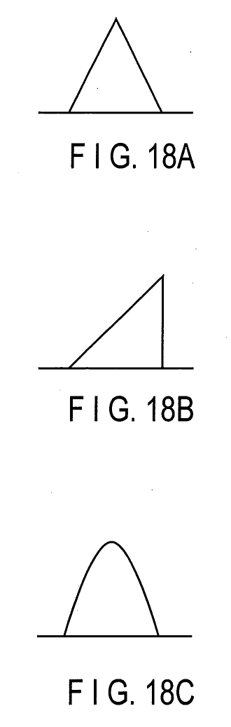

A third variant will be described below. In the foregoing one embodiment, the pulse drive current I is a rectangular wave pulse signal. However, the pulse signal is not limited to a rectangular wave one. For example, the pulse drive current I may have a triangular waveform as shown in FIG. 18A, a saw-toothed waveform as shown in FIG. 18B or a curved waveform as shown in FIG. 18C.

[Fourth Variant]

A fourth variant will be described below. In the foregoing one embodiment, the light source controller 16 specifies three peak current values Ia, Ib and Ic of rectangular wave pulse drive currents I.

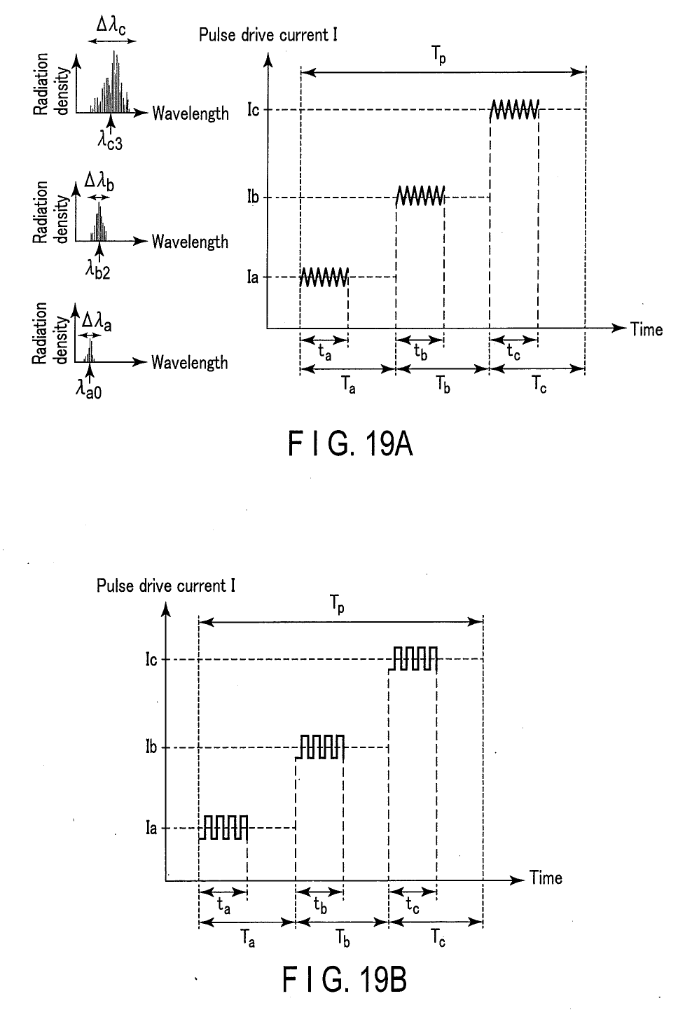

On the other hand, in the fourth variant, the pulse drive current I may have a sine waveform as shown in FIG. 19A or the pulse drive current I may have a rectangular waveform as shown in FIG. 19A. In the fourth variant, an average current value of alternating currents of the sine wave or rectangular wave pulse drive current I is specified. The control of the light of the LD 11-1 is performed by controlling the number of crests of the sine or rectangular wave or the light-emission time of the LD-11.

Therefore, the light source controller 16 supplies the LD 11-1 with a plurality of alternating drive currents including the average current value that varies within exposure time Tp of the image sensor 19 to cause the LD 11-1 to emit, for example, three pulsed lights Q1, Q2 and Q3. The light source controller 16 controls the average current value of a plurality of alternating drive currents such as sine wave or rectangular wave drive currents in such a manner that the combined wavelength spectrum width Δλabc (=Δλa+Δλb+Δλc) of combined pulsed light into which three pulsed lights Q1, Q2 and Q3 are combined, is made greater than each of the wavelength spectrum widths of the three pulsed lights Q1, Q2 and Q3.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope apparatus comprising:
a laser diode;
an illumination section which irradiates an observation target with laser light emitted from the laser diode as illumination light;
an image sensor which images the observation target irradiated with the illumination light by the illumination section; and
a light source controller which supplies the laser diode with different drive currents in sequence within exposure time of the image sensor to emit a plurality of laser lights in sequence from the laser diode.

2. The endoscope apparatus according to claim 1, wherein the light source controller controls the drive currents in such a manner that a combined wavelength spectrum width of combined laser light obtained by combining the emitted laser lights with the exposure time is made greater than a wavelength spectrum width of each of the laser lights.

3. The endoscope apparatus according to claim 1, wherein the light source controller supplies the laser diode with a plurality of pulse drive currents of different peak current values as the drive currents in sequence within exposure time of the image sensor to emit a plurality of pulsed lights in sequence from the laser diode and controls the peak current values of the pulse drive currents in such a manner that a combined wavelength spectrum width of combined pulsed light obtained by combining the pulsed lights within the exposure time is made greater than a wavelength spectrum width of each of the pulsed lights.

4. The endoscope apparatus according to claim 3, wherein the light source controller supplies the laser diode with a plurality of alternating drive currents including different average current values as the drive currents in sequence within exposure time of the image sensor to emit a plurality of pulsed lights in sequence from the laser diode and controls the average current values of the alternating drive currents in such a manner that a combined wavelength spectrum width of combined pulsed light obtained by combining the pulsed lights within the exposure time is made greater than a wavelength spectrum width of each of the pulsed lights.

5. The endoscope apparatus according to claim 1, wherein the light source controller controls the drive currents in such a manner that a difference in central wavelength between adjacent two of the laser lights or the pulsed lights with respect to a wavelength axis is equal to or greater than a predetermined wavelength difference based on a wavelength spectrum width of the two of the laser lights or the pulsed lights.

6. The endoscope apparatus according to claim 3, wherein the light source controller controls the peak current values of the pulse drive currents in such a manner that a difference in central wavelength between adjacent two of the laser lights or the pulsed lights with respect to a wavelength axis is equal to or greater than a predetermined wavelength difference based on a wavelength spectrum width of the two of the laser lights or the pulsed lights.

7. The endoscope apparatus according to claim 4, wherein the light source controller controls the average current values of the alternating drive currents in such a manner that a difference in central wavelength between adjacent two of the laser lights or the pulsed lights with respect to a wavelength axis is equal to or greater than a predetermined wavelength difference based on a wavelength spectrum width of the two of the laser lights or the pulsed lights.

8. The endoscope apparatus according to claim 3, wherein the light source controller controls the peak current values of the pulse drive currents in such a manner that a wavelength region of a wavelength spectrum of one of the pulsed lights is not included in a wavelength region of a wavelength spectrum of the other pulsed lights

9. The endoscope apparatus according to claim 6, wherein the light source controller controls the peak current values of the pulse drive currents in such a manner that a difference in central wavelength between the adjacent two of the pulsed lights with respect to the wavelength axis is a wavelength difference that is equal to or greater than half of a wavelength spectrum width of one of the adjacent two of the pulsed lights, a wavelength spectrum width of which is smaller.

10. The endoscope apparatus according to claim 9, wherein the light source controller controls the peak current values of the pulse drive currents in such a manner that a difference in central wavelength between the adjacent two of the pulsed lights with respect to the wavelength axis is a wavelength difference that is equal to or greater than halves of wavelength spectrum widths of the adjacent two of the pulsed lights.

11. The endoscope apparatus according to claim 6, wherein the light source controller controls the peak current values of the pulse drive currents in such a manner that the pulse drive currents to cause mode hopping in which an oscillation mode hops are specified as mode hopping current values when the pulse drive currents supplied to the laser diode are varied continuously, and the peak current values of the pulse drive currents are specified over the mode hopping current values.

12. The endoscope apparatus according to claim 11, wherein the light source controller specifies, as the peak current values, one or both of a lasing threshold neighboring current value which is close to a lasing threshold value of the laser diode and which is equal to or larger than the lasing threshold value and a rated current neighboring current value which is close to a maximum rated current value of the laser diode and which is equal to or smaller than the maximum rated current value.

13. The endoscope apparatus according to claim 12, wherein the light source controller specifies, as the peak current values of the pulse currents, the lasing threshold neighboring current value, the rated current neighboring current value, and an intermediate current value between the lasing threshold neighboring current value and the rated current neighboring current value.

14. The endoscope apparatus according to claim 13, wherein the light source controller specifies the peak current values of the pulse drive currents at an equal interval from the lasing threshold neighboring current value and the rated current neighboring current value in a current range therebetween.

15. The endoscope apparatus according to claim 13, wherein the light source controller specifies the intermediate current value in such a manner that a central wavelength of the pulsed light exists at an equal interval from a central wavelength of the pulsed light whose lasing threshold neighboring current value is a peak current and a central wavelength of the pulsed light whose rated current neighboring current value is a peak current in a wavelength range therebetween.

16. The endoscope apparatus according to claim 3, wherein the light source controller includes a light control circuit which makes a light control for the laser diode, and the light control circuit makes the light control by performing pulse width control to control pulse widths of the pulse drive currents within the exposure time.

17. The endoscope apparatus according to claim 3, wherein the light source controller includes a light control circuit which makes a light control for the laser diode, and the light control circuit makes the light control by specifying a pulse width control period to control a pulse width of each of the pulse drive currents within the exposure time and controlling a duty ratio that is a light-emission time ratio of the laser diode in the pulse width control period.

18. The endoscope apparatus according to claim 17, wherein the light control circuit increases the duty ratio of the pulse drive currents whose peak current values are small to increase an intensity of laser light emitted from the laser diode, and decreases the duty ratio of the pulse drive currents whose peak current values are large to decrease an intensity of laser light emitted from the laser diode.

19. The endoscope apparatus according to claim 18, wherein the light control circuit makes the light control by making the pulse width control period variable in accordance with the intensity of laser light emitted from the laser diode and making a length of the pulse width control period variable in proportion to the amount of the peak current values if the intensity of laser light is larger than a predetermined light intensity.

20. The endoscope apparatus according to claim 3, wherein the light source controller includes a light control circuit which makes a light control for the laser diode, and the light control circuit makes the light control by supplying the laser diode with the pulse drive currents whose peak current values are equal for each predetermined period within the exposure time and performing pulse number control to control the number of pulse drive currents during the predetermined period.

21. The endoscope apparatus according to claim 20, wherein the light control circuit sets a pulse number control period to perform the pulse number control and controls the number of pulse drive currents whose peak current values are equal.

22. The endoscope apparatus according to claim 21, wherein the light control circuit increases the number of pulse drive currents whose peak current values are small to increase an intensity of laser light emitted from the laser diode, and decreases the number of pulse drive currents whose peak current values are large to decrease an intensity of laser light emitted from the laser diode.

23. The endoscope apparatus according to claim 22, wherein the light control circuit makes the light control by making the pulse number control period variable in accordance with the intensity of laser light emitted from the laser diode and making a length of the pulse width control period variable in proportion to the amount of the peak current values if the intensity of laser light emitted from the laser diode is larger than a predetermined light intensity.

24. The endoscope apparatus according to claim 23, wherein the light source controller includes a storage circuit which stores the peak current values of the pulse drive currents.

25. The endoscope apparatus according to claim 17, wherein the light source controller includes a storage circuit which stores information about the pulse width control period for an intensity of the laser light emitted from the laser diode, and the light control circuit performs the pulse width control based on the information stored in the storage circuit.

26. The endoscope apparatus according to claim 20, wherein the light source controller includes a storage circuit which stores correlation information of the pulse number control of the pulse drive currents whose peak current values are equal for an intensity of the laser light emitted from the laser diode, and the light control circuit performs the pulse number control based on the correlation information stored in the storage circuit.

27. The endoscope apparatus according to claim 1, comprising a plurality of laser diodes which emit laser lights of different oscillation wavelengths.
